(19) United States
(12) Patent Application Publication
(10) Pub. No.: US 2020/0015734 A1
(43) Pub. Date: Jan. 16, 2020

(54) “One Stop Shop” for Prostate Cancer Staging using Imaging Biomarkers and Spatially Registered Multi-Parametric MRI

(71) Applicant: Rulon Mayer, Garrett Park, MD (US)

(72) Inventor: Rulon Mayer, Garrett Park, MD (US)

(21) Appl. No.: 15/831,989

(22) Filed: Dec. 5, 2017

(51) Int. Cl. A61B 5/00; A61B 5/055; G01R 33/56; G01R 33/50; G01R 33/563; G06T 3/40; G06T 3/00; G06T 3/20; G06T 7/00; G06T 7/90; G06T 7/62

(52) U.S. Cl. CPC A61B 5/4381; G06T 2207/30242; G01R 33/5601; G01R 33/5608; G01R 33/50; G01R 33/56341; G06T 3/4038; G06T 3/0068; G06T 3/20; G06T 7/0012; G06T 7/90; G06T 7/62; G06T 2207/10096; G06T 2207/30081; G06T 2207/30096; G06T 2207/20081; G06T 2207/20224; A61B 5/055

(57) ABSTRACT

The purpose of this embodiment is to describe a “one stop shop” for staging prostate cancer and a novel application of supervised target detection algorithms to spatially registered multiparametric MRI images in order to non-invasively detect, locate, and score prostate cancer at the voxel level and measure the tumor volume and assign color to the spatially registered MRI to highlight and display tumors, and detect metastases (specifically in the seminal vesicle). To test the approach advanced by the embodiment, a retrospective study analyzes MRI from 26 patients that had also undergone robotic prostatectomy. Whole-mount sections were stained for histopathologic evaluation and matched to the MRI. The stained sections were independently reviewed by pathologists. All slices of various types of MRI were spatially registered and stitched together. Signatures or image-based biomarkers from registered multiparametric MRI training sets were extracted. The untransformed and “whitened-dewhitened” transformed signatures (based on the statistics of the normal prostate) from a battery of Gleason scores were applied to the stitched hypercubes. Each voxel in the supervised target map was polled to find the signature that achieved the highest Gleason score likelihood. The Gleason scoring and volume measurements were quantitatively validated by comparing the results from 10 patients with prostate adenocarcinoma to the pathologist's assessment of the histology. High correlation between supervised target detection using “whitened-dewhitened” transformed signatures and histology was observed (p<0.02). Assigning red, green, and blue to the registered MRI hypercubes effectively displays tumors relative to normal prostate tissue. With only minor modifications, supervised target detection and transformation of target signatures and color display may be used to find metastases, specifically to the seminal vesicles. This novel application of supervised target detection algorithms to spatially registered multi-parametric MRI non-invasively detects, locates, and scores prostate cancer at each voxel level and measures the tumor volume.

### CROSS REFERENCE

This application claims the priority benefits of U.S. Provisional Application No. 62/430,692, EFS ID 27711893, Confirmation Number 7284, filed Dec. 6, 2016, the contents of which are hereby incorporated by reference in its entirety.

### FEDERAL SUPPORT AND GOVERNMENT RIGHTS

Work was only partially supported by the Murtha Cancer Center Comprehensive Research (MCC)—Award No. HU0001-14-1-0010, Project No. PRS-12-2804 awarded by Uniformed Services University. The support finished financing this effort March, 2016. Most of the work was generated before and after the grant duration over a period of 19 years. The government has certain rights in this invention.

The inventor (Rulon Mayer) failed to find any comparable patents in the United State Patent Data base that employ untransformed and/or transformed signatures or image-based biomarkers inserted into multispectral supervised target algorithms to score tumors or measure their size nor employ colors (described in this Specification) using multiparametric MRI to highlight tumors. However, there is a body of research that examined some related issues that pertain to this patent application. This Background Section will cite and summarize the relevant literature and describe some of their limitations.

### BACKGROUND

Prostate cancer (PCa) is the most common malignancy and the leading cause of cancer-related death in men in the United States [1]. Gleason Score (GS) is a validated predictor of PCa disease progression, mortality, and outcome [2, 3]. The GS, determined through biopsies, however, suffers from significant interobserver variability, potential for sampling error that can lead to false negatives or underestimate the severity of the disease, and can differ from those determined through radical prostatectomy [4, 5] and between immediate repeat biopsies [6]. Automatically and non-invasively detecting the GS with high accuracy from diagnostic Magnetic Resonance Imaging (MRI), therefore, could significantly impact clinical decision making and treatment options for patients and spare them from invasive biopsies and their accompanying pain and possible complications. Specifically, non-invasive MRI tumor detection could help manage patients with high persistent prostate specific antigen levels (PSA) but negative needle biopsy. The MRI could also be a valuable adjunct for patients with low grade, low volume PCa who are undergoing active surveillance, and for monitoring of potential relapse or recurrence of PCa following therapy. PCa aggressiveness assessment by noninvasive and highly accurate means are needed to enhance the quality of patient care and improve outcomes.

Considerable effort and hope has been expressed [7] in the literature for the potential benefit of exploiting the distinguishing MRI features of the diseased prostate. Prostate tumors empty and fill contrast material due its high vasculature to support the elevated nutritional needs of the tumor and is manifested in the contrast material time evolution MRI such as Dynamic Contrast Enhancement (DCE). The high cellular density for prostate tumors impedes movement of water molecules and is seen in low Apparent Diffusion Coefficient (ADC) but relatively higher values for the high B (high field gradient) for Diffusion Weighted Images (DWI). Some researchers [8-11] used a single modality such as ADC, DWI, DCE, T2 to detect and localize the tumors within a prostate. In these cases, statistical averages of a given MRI modality over a given region of interest (ROI) delineated by the radiologist are computed and used as a metric of disease. The measurements were compared to the “gold standard” of assessment of histology slides taken from prostatectomy specimens processed in MRI-based molds. More commonly, other researchers [12-24] have combined two or more of the modalities in the Multi-Parametric (MP) MRI approach to detect and localize the disease. The use of multiple sets of data tends to increase the sensitivity and specificity for finding the disease. The two or more sets of MRI are often not spatially registered at the pixel level to each other. However, the multiple MRI images are correlated with each other, and the statistics of the region of interest (ROD are separately determined by the researchers and compared to the pathologist's evaluation of the histology from tissue taken from radical prostatectomy. Increasing number of modalities generally elevate the sensitivity and specificity. Similarly, researchers [25-28] used single modalities such as ADC, DWI, DCE, T2 to Gleason score and assess the disease. They compare the statistical metric with the histology assessment of the Gleason score or expected tumor aggressiveness. The use of multiple sets [29-35] of data generally increases the sensitivity and specificity for scoring the disease.

A severe limitation of previous studies is the lack of a consistent, coherent approach that can be applied to all clinics and patients to support protocols. Machine language approaches retrain to on a new set of images in order handle varying global clinical situations such as different field sizes, pulse sequences et al. Such retraining is time consuming and limits clinical applications and studies. Conventional approaches do not operate at the voxel level, are often only qualitative, and can depend on the observations of a trained radiologist.

The embodiment analyzes the spectral distribution only and departs from more standard Computer Aided Diagnosis (CAD) algorithms that depend solely on spatial analysis of a specific MP MRI modality. In addition, this proposed research does not emulate radionomics that rely on extracting spatial features from a single image modality. Most approaches that discriminate between cancer and normal tissue or Gleason score depend on spatial processing of an image and that may assess textures such as the local roughness, smoothness etc. in order to distinguish cancer and normal tissues or evaluate the tumor aggressiveness. Texture-based imaging features in conjunction with machine learning-based classifications have been applied for classifying malignant from noncancerous prostate tissues [35, 42, 43]. Texture and spatial processing requires setting fixed spatial windows to assess the local environment. Because these windows are fixed, the texture and spatial processing can miss detecting tumors that can vary considerably in size, especially are vulnerable to missing small lesions.

Current methods for assessing the location of prostate lesions divide the prostate into only 20 to 50 segments [18, 20, 21]. These methods fail to fully exploit MRI's high spatial resolution. Furthermore, these current techniques do not evaluate the variable aggressiveness inside the tumor and only summarize the lethality with a single metric, despite the considerable heterogeneity inside the tumor. In addition, the parameters used in the malignancy probability [16] and Composite Biological Score (CBS) [23] are fixed from patient to patient in order to determine whether a voxel is a lesion or normal tissue. These parameters may vary for each patient and these current techniques may not offer a robust solution for assessing patients.

This embodiment aims to buttress non-invasive staging of prostate tumors by also non-invasively finding metastases, a critical component for staging a patient with possible prostate tumor burden, along with measurements of tumor volume measurement and tumor aggressiveness. A staging system is a standard way for the cancer care team to describe how far a cancer has spread. The most widely used staging system for prostate cancer is the American Joint Committee on Cancer (AJCC) TNM system. The TNM system for prostate cancer is based on five key pieces of information: the extent of the main (primary) tumor (T category), whether the cancer has spread to nearby lymph nodes (N category), whether the cancer has metastasized to other parts of the body (M category), the PSA level at the time of diagnosis, the Gleason score, based on the prostate biopsy (or surgery). Unlike current practice, this embodiment develops a “one-stop shop”, non-invasive procedures using MP-MRI to determine most of the essential components for staging, namely tumor volume measurements, cancer spread to nearby lymph nodes, and Gleason Scoring. Future research should determine metastases to more distant sites. The approaches advanced in this embodient do not address PSA measurements.

Comparing the scoring results using image-based biomarkers and supervised target detection with other approaches is currently problematic. The embodiment offers the first description of this technique. In the future after further validation this technique will be compared with other approaches. For example, Pi-Rads v2 [41] is relatively new (2016) and requires experienced and specially trained radiologists to examine the entire prospective tumor, rather than evaluate every voxel within the tumor. Currently Pi-Rads v2 assessments are relatively rare compared to the more conventional histological determination of Gleason score tumor volume. The embodiment, however, employs relatively few radiologists. Other approaches use self-training and learning approaches [17, 19, 55] to detect tumors.

### SUMMARY

This embodiment describes non-invasive prostate tumor staging using spatially registered multi-parametric MRI. This novel embodiment adapts supervised target algorithms from hyperspectral images generated for surveillance and defense applications where the problem of discriminating a “target” against a complex “cluttered” environment is routine. Instead of using wavelengths or spectroscopy, we employ imaging data from T1, T2, ADC, and DWI to distinguish tumor (targets) from normal prostate tissue (backgrounds). These data can be viewed as similar to multispectral data employed for target detection from drone imagery. The algorithms discussed in this study use multispectral tumor “signatures” whose vector elements are composed of the MRI parameters. These “signatures” can be viewed as novel image-based biomarkers that characterize the tumor and its potential aggressiveness, as described by the Gleason score. The signatures are generated from a training set of images that compare the spatially registered multi-parametric MRI with the Gleason score of a pathologist's assessment the histology slices taken from patients that underwent radical prostatectomy. The embodiment applies supervised target algorithms (with the signatures) to spatially registered MRI in order to non-invasively find and score and measure the size of prostate tumors. Unlike most other approaches, the image processing algorithms (see Background in the Patent Application) described in this proposal use spatially registered MRI. In addition, this embodiment describes the statistical transformation of these signatures using the “Whitening-DeWhitening” transform to handle varying global clinical situations.

Another byproduct of using registered images is the ability to use a color display to enhance the presence of tumors. The display is not a “pseudo” color but instead is a natural byproduct of treating each voxel as a “vector.” Prostate tumors have fast kinetics (quickly fill and empty) and low diffusion due to high cellular density. In the case of the color image, only three components are used instead of all seven components. That is, three image components, K^{trans} or Washout, DWI-High B, and ADC are judiciously assigned as red, green, and blue channels, respectively. Using such a choice, the tumor appears as yellow due to the high washout rate (red), high DWI-Hi B (green), and low ADC (blue). Nevertheless, the prostate tumor is clearly displayed for easy viewing by the radiologist and therapist. In addition, the tumor heterogeneity is clearly highlighted in the image.

The application of supervised target detection and color displays described in this embodiment can also find metastases in seminal vesicles that feed the prostate, a critical component in the staging of prostate cancer.

### BRIEF DESCRIPTION OF THE DRAWING (FIGURE CAPTIONS)

FIG. 1A is an image of ADC of prostate. Darkened area shows tumor. FIG. 1B is an image of kep or washout from DCE. Bright area show tumor. FIG. 1C is a scatter plot of registered ADC vs. kep. appear as bright points are high kep and low ADC or tumor. FIG. 1D. is an image of bright points from FIG. 1C scatter plot are superimposed onto ADC image.

FIG. 2. is a two-dimensional scatter plot of MRI-Parameter 2 vs. MRI-Parameter 1 schematic showing ACE detection decision surface cone, normal prostate (background) pixels, tumor (target) pixels, target signature, transformed signature, false alarms, decision surface.

FIG. 3A, FIG. 3B, FIG. 3C, FIG. 3D are images illustrating the analysis of Dynamic Contrast Enahancement image taken from Patient #11. FIG. 3A is an image of an ADC slice. Dark area shows low diffusion and possible tumor FIG. 3B is an image of a time profile from tumor taken from a single tumor pixel shown in dark area of FIG. 3A and decay for times longer than 100 seconds. FIG. 3C is a time profile from normal prostate taken from a single tumor pixel shown in bright area in FIG. 3A. and shows growth in contrast. FIG. 3D is an image of washout showing decay in time profile from analyzing DCE. Bright areas show elevated decay rate and tumor vascularity

FIG. 4A is an image of a hypercube displayed as red, green, blue assigned to kep, DWI-High b value, ADC FIG. 4B is an image of an expanded view of a single slice from FIG. 4A. FIG. 4C is an image of mosaic of kep FIG. 4D is an image of an expanded view of a single slice from FIG. 4C. FIG. 4E is an image of mosaic of the prostate mask FIG. 4F is an image of expanded view of a single slice from FIG. 4E.

FIG. 5A is an image of k or Washout, note intense area for tumor, FIG. 5B is an image of DWI High B, note intense area for tumor, FIG. 5C is an image of ADC, note dark area for tumor. FIG. 5D is an image of Color generated by assigning red channel to Washout, green to DWI High B, blue to ADC. FIG. 5E is an image of histology slice most closely matches MRI, tumor outlined. FIG. 5F is an In-Scene ACE Detection map shown as False color image.

FIG. 6A is an image of RGB (red=Washout, kₑₚ, green=DWI-Hi B, blue=ADC) from Patient #11 where the tumor is displayed in yellow in the RGB image but as a bright region in the black and white image FIG. 6B is a histology image taken from Patient #11, Slice #4. The tumor is outlined by a pathologist. FIG. 6C is an image of RGB (red=Washout, kₑₚ, green=DWI-Hi B, blue=ADC) from Patient #11. The red areas denote the tumor in MP_MRI image (239 pixels) FIG. 6D is a histology image taken from Patient #11, slice #4. The red areas denote the tumor in histology image (323,196 pixels). Accounting for spatial resolution results in a scaled (to 1 mm per pixel) pixel number of 145 pixels.

FIG. 7A, FIG. 7B, FIG. 7C, FIG. 7D provide an outline for non-invasive Gleason scoring. FIG. 7A is a Tumor Signature characterize tumor showing values for MP-MRI. FIG. 7B is a schematic of normal and tumor related signatures FIG. 7B is a schematic of signatures inserted into ACE/SAM detection mappers. FIG. 7C is a schematic of hypercube with tumor shown FIG. 7D is a schematic for the highest ACE/SAM score or a weighted average of the top two scores of each pixel inside tumor is sampled FIG. 8A is an image of Prostate displayed as color image where red is kep green is DWI-High B blue is ADC FIG. 8B is an image of Gleason scoring inside tumor using Maximum SAM with Whitened-DeWhitened signatures FIG. 5C is an image of Gleason Scoring Weighted SAM using Whitened-DeWhitened signatures.

FIG. 9. is a plot of Tumor Volume taken from Histogram vs Volume from MRI (R=0.94).

FIG. 10 is a plot of the average Gleason score within a tumor using the top scorer and no transformation, top scorer with transform, weighted top 2 scorers no transform and weighted top 2 scorers with transform. The vertical and horizontal axis record the numerical Gleason scheme. Conventional Gleason scores are superimposed on each axis.

FIG. 11A is an image of metastases to seminal vesicle using color scheme of red, green, blue applied to registered Washout, DWI (High B), and T2, respectively. FIG. 11B is an image of an expanded region of FIG. 11A showing metastases in seminal vesicle as yellow in color but as a bright area in the black and white image.

### LIST OF ABBREVIATIONS

3D Three Dimensional
ACE Adaptive Cosine Estimator
ADC Apparent Diffusion Coefficient
AUC Area Under Curve
CBS Composite Biological Score
Cₚ Plasma Concentration
Cₜ Extracellular Extravascular Concentration
COTS Commercial Off The Shelf, for example IDL
CS Custom Software, built using IDL for example
CVM CoVariance or Clutter Matrix
DCE Dynamic Contract Enhancement
DWI Diffusion Weighted Image
ENVI Commercial Software for processing multi spectral images
GS Gleason Score
IDL Interactive Development Language
High-B Largest Magnetic Field Gradient for diffusion weighted images
IRB Internal Review Board
Kₑₚ Extracellular Extravascular Plasma Filling Constant
K^{trans} Transfer Constant, Emptying Constant
m Average Background (normal prostate) vector
mpMRI Multi-parametric Magnetic Resonance Image
MRI Magnetic Resonance Image
NCI National Cancer Institute
NIH Nation Institutes of Health
PCa Prostate Cancer
P p-value
PSA Prostate Serum Assay
RGB Red, Green, Blue
ROC Receiver Operator Characteristic
ROI Region of Interest
R Correlation Coefficient
S Signature or Biomarker Vector
SAM Spectral Angle Mapper
SCR Signal to Clutter Matrix
T1 Longitudinal Relaxation Time
T2 Latitudinal Relaxation Time
t Time
rTime, dummy variable
TCIA The Cancer Imaging Archive
TRUS Trans Rectal Ultra Sound
UMD University of Maryland
UPenn University of Pennsylvania
WRNMMC Walter Reed National Military Medical Center
x Pixel Vector

### DETAILED DESCRIPTION

### I. Advantages

The general approach is less dependent on the expertise of a trained radiologist. Employing the Whitening-DeWhitening transform permits flexibility and offers a robust approach to handling a variety of patients and clinical situations by transforming each signature for every patient. The signatures can be part of a library and they can then be transformed and tailored to a specific patient, imaging conditions, and imager calibrations etc. The preliminary study in support of the embodiment finds that transforming signatures are essential for achieving higher sensitivity. In this approach we do not weight one parameter higher than another, but rather use 7 different MRI derived data hypercubes. The transforms use the multispectral statistics of normal prostate to help transform the signatures. The algorithms are consistent for all patients and clinical situations thereby aiding protocols. The described methodology is therefore more robust and flexible than conventional approaches. Non-invasive approaches is also less burdensome for the patient and may result in fewer deleterious conditions such as hemorrhaging from needle biopsy and less patient discomfort.

As noted in the Background Section, the most common approach is to manipulate or spatially process a single MR image to generate features or textures. These conventional approaches set a spatial window size. Tumors, however, can have wide variety of sizes. Using the spectral domain to analyze and detect tumors has the virtue of begin able to handle any size of tumor due to the absence of the limiting spatial parameters. In addition, the application of supervised target detection and using transformed signatures examines greater number of voxels (roughly 10,000 prostate voxels) relative to conventional approaches for evaluating prostate tumors. Furthermore, the embodiment detects the Gleason Score for each voxel inside the tumor unlike current techniques that only summarize the lethality with a single metric, despite the considerable heterogeneity inside the tumor. In addition, the parameters used in the malignancy probability [16] and Composite Biological Score (CBS) [23] are fixed from patient to patient in order to determine whether a voxel is a lesion or normal tissue. These parameters may vary for each patient and for different clinical situations (fields, pulse sequences) and therefore may not offer a robust solution for assessing patients and employed for protocols.

Normally, tumors appear in an MRI as an asymmetric or anomalous difference (darker or lighter) relative to normal tissue. Such differences may be difficult to detect for the untrained eye. The embodiment exploits the vector nature of each voxel and tumor physiology by applying a simple coloring scheme to highlight the tumor (appearing as yellow) relative to the normal tissue (bluish). Such a coloring scheme also readily displays the tumor heterogeneity to help denote the possibly more aggressive parts of the tumor and aid the clinician.

The supervised target detection and color display method described here can be extended to other parts of the body. It is expected that target detection and color display should enhance tumor delineation for other primary tumors such as brain malignancies and detecting metastases, such as involvement in the seminal vesicles. This study will lead to automatic and non-invasive detection of GS from diagnostic MRIs that could significantly impact clinical decision making, aid tumor staging, and treatment options for patients. It may also help less experienced readers perform at the level of an expert, more accurately target MR-guided biopsies, and enable focal therapies.

### II. General Approach

Prostate cancer (PCa) and the normal prostate gland exhibit differences in their physiology, and these differences are manifested in MRI images. In PCa, the water molecules motion is impeded by the high cellular density resulting in lower diffusion for the protons detected in the MRI. Therefore PCa exhibits a relatively reduced value in Apparent Diffusion Coefficient (ADC) and appears darker relative to normal prostate in the ADC image. The ADC image results from fitting a series of Diffusion Weighted Images (DWI) exposed to varying levels of magnetic field gradients (proportional to a quantity B). The DWI with the highest gradients (High B) tends to relatively elevate the values of the tissues with lower diffusion coefficients. In this case, the PCa will appear relatively brighter in the DWI-High B (B=1000 sec/mm²) image compared to normal prostate tissue. In addition, tumors are rapidly replicating and growing and, therefore, need nutrients supplied by blood transported through primitive vasculature. Contrast material injected into the patient can preferentially diffuse to the lesion. The contrast material rapidly fills the tumor, permeates the vascular walls, enters the extra cellular space (but not the tumor cells), and then reenters the blood stream. The Dynamic Contrast Enhancement (DCE) is a series of T1 images showing the time evolution of each pixel. The DCE shows tumors (recorded by time sequence of T1 images and values) rapidly fill (characterized through fitting by a filling rate K^{trans}) and empty (characterized through fitting by a “washout” rate Kₑₚ). Contrast material in normal tissues (and associated T1 values) tend to slowly rise and do not significantly empty throughout the time period of the imaging. Tumor and normal tissue can be distinguished from each other by mathematically fitting the time profiles for each pixel in the DCE and finding the rate of return of the contrast material from the extravascular space to the plasma.

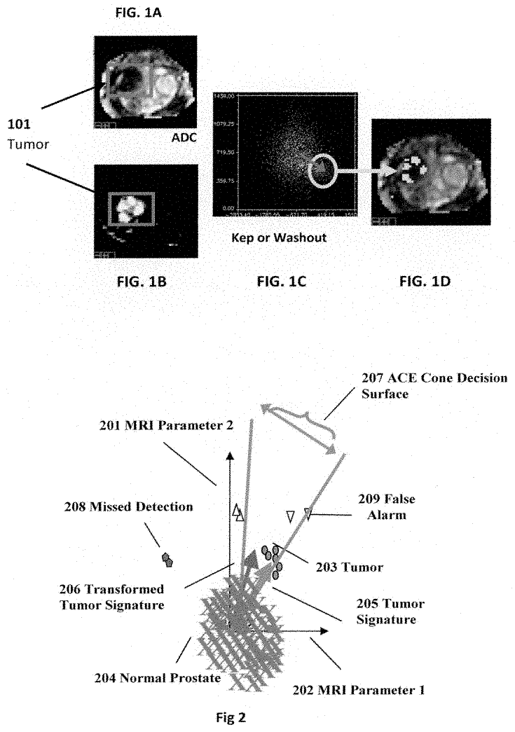

The embodiment combines the information from images of diffusion, T2, and time evolution of contrast material to help objectively discriminate tumors from normal prostate. A distinguishing aspect of this study is that all MRI modalities (7 in this study) are registered to each other at the voxel level. Such an arrangement treats each voxel in the image as a component of a vector (in 7 dimensions). To illustrate the concept, FIG. 1A shows an ADC image and FIG. 1B shows kep (or washout) image. FIG. 1A shows a tumor 101 as a darkened region in the ADC (low diffusion) and also FIG. 1B shows the same tumor 101 but as a brighter region in the washout or elevated clearance rates derived from the DCE mage set. A scatter plot of washout vs. ADC of each point in the prostate is shown in FIG. 1C. Most of the points in the plot are distributed over a large area. However, a region with low ADC but larger washout values (lower left quadrant) is highlighted as a brighter in region in the black and white image. Note that these brighter areas are located a certain distance and angle away from the centroid of the scatter plot of normal prostate. In other words, a vector (arrow) can be extended from the normal prostate centroid to the centroid of the tumor (lighter points) and has a magnitude and direction. The lighter points in a small corner of the FIG. 1C are mapped into the ADC image (FIG. 1D) and these points appear inside of the tumor demonstrating the correlation between the signature vector and the tumor. This embodiment uses seven modalities, not two shown as in the illustrative example, and should greater discriminate targets relative to background.

The embodiment registers MRI modalities such as T1, T2, ADC, DWI to distinguish tumor (targets) from backgrounds instead of wavelengths or spectroscopic data. These data can be viewed as similar to multispectral data employed for target detection from drone imagery. The algorithms discussed in this embodiment use multispectral tumor signatures and do not employ arbitrary fitting parameters, and they instead simply combine all MRI modalities. Unlike other medical approaches, this embodiment treats each voxel as a vector composed of MRI modality, rather than a scalar value. The embodiment incorporates the adaption of supervised target algorithms from hyperspectral images generated for surveillance and defense applications where the problem of discriminating a “target” against a complex “cluttered” environment is routine. This research in support of this embodiment develops, tests, and applies the algorithms to a spatially registered MRI in order to non-invasively find and score prostate tumors and also measure their volume which will serve to guide treatment recommendations for prostate cancer. The multispectral feature for the spatially registered MRI and tumor physiology (such as reduced diffusion of water molecules and elevated contrast material kinetics) are exploited in this embodiment by assigning three of the registered images to red, green, and blue colors and using the resulting combined “true color” to highlight and display the tumor. This embodiment relies on the spectral distribution alone and departs from more standard CAD algorithms that depend solely on spatial analysis. The Gleason Scores and prostate tumor measurements are compared to the “gold standard”, namely the results from an evaluation by the pathologists of histology slices taken from whole mount prostates that have been resected from the patient. This newer approach and testing do not compare Gleason Scoring to the newer, less tested PI-RADS approach derived from inspecting MRI. [41].

### III. Materials and Methods

A. Mathematical Background:

The image processing programs developed previously for Defense and Surveillance applications were modified in this embodiment. These programs generate registered hypercubes composed of MRI images such as T1, T2, DWI, and ADC images. Additional modalities were generated for prostate cancer using the Dynamic Contrast Enhancement images.

MRI modalities such as T1, T2, ADC (Apparent Diffusion Coefficient), DWI (Diffusion Weighted Images) were spatially registered to distinguish tumor (targets) from backgrounds instead of wavelengths taken from spectroscopic data. Prostate cancer is highly vascularized (contrast material quickly fills and empties) but also has high cellular density and reduced diffusion (low ADC, low T2). Specifically, the washout for contrast material using the Dynamic Contrast Enhancement (DCE) is calculated for each pixel and used to help find the highly vascularized tumor. These data can be viewed as similar to multispectral data employed for target detection from drone imagery. The algorithms discussed in this study do not employ parameters and instead simply combines all MRI modalities. This approach relies on the spectral distribution alone and departs from more standard CAD algorithms that depend solely on spatial analysis.

FIG. 2 schematically shows the Supervised Target Detection algorithm in two dimensions (not 7 for simplicity). FIG. 2 is a scatter plot for voxel values of MRI Parameter 2 201 plotted against MRI Parameter 1 202. Each voxel in the image is associated with a vector (magnitude, angle or direction) rather than a scalar (single value) quantity that originates from the center of the background, or voxels assigned to the normal prostate. Targets are characterized by their signature or detected intensity as a function of wavelength or MRI modality in this medical application. Following training, tumor or target voxels 203 and normal prostate (background) 204 are identified. The vector extending from the center of the background 204 to the center of tumor pixels 203 is the tumor or target vector or “signature” 205. Each modality contributes some information to each voxel. Following “training” or identifying the target signature or vector from comparing with histological analysis from slices derived from radical prostatectomy. Tumor or target signatures 205 may be transformed into a more suitable vector 206 using “Whitening-DeWhitening” (described below) to handle varying conditions. The target signatures are inserted into a supervised target detection algorithm such as Adaptive Cosine Estimator (ACE) and each pixel [36, 37] is determined to be background or target. ACE uses the conical hyperspace decision surface 207 to find pixel alignment or angular deviation. Tumor or target pixels that reside outside the conical surface 207 are identified as missed detections 208. Background pixels residing inside the decision surface 207 are False Alarms 209. Mathematically, the ACE score at a given voxel i that has a seven-component vector containing the values of all MRI modalities is given by

$$ACE ( i ) = \frac{{( {x}_{i} - m )}^{T} {CVM}^{- 1} ( S - m )}{\begin{matrix}{( {( {x}_{i} - m )}^{T} {CVM}^{- 1} ( {x}_{i} - m ) )}^{1 / 2} \\ {( {( S - m )}^{T} {CVM}^{- 1} ( S - m ) )}^{1 / 2}\end{matrix}} \qquad ( 1 )$$

In Equation 1 (and the rest of this embodiment) m is the background (normal prostate) or mean value for all 7 modalities and is a 7 component vector, CVM is the covariance or clutter matrix (7×7), and S is the tumor signature (7 component vector).

Matrix multiplication is assumed in this equation as well as the rest of the equations in this embodiment. The superscript T denotes the transpose matrix operation. The superscript −1 denotes a matrix inversion operation

$$SAM ( i ) = \frac{{( {x}_{i} )}^{T} ( S )}{{( {( {x}_{i} )}^{T} ( {x}_{i} ) )}^{1 / 2} {( {( S )}^{T} ( S ) )}^{1 / 2}} \qquad ( 2 )$$

The pilot study for this embodiment detected cancers from a number of patients. Each of the patients of various sizes were imaged with possibly small differences in pulse sequences that could globally affect the MRI values and ability to detect targets. The Whitening-DeWhitening is the affine transform that minimizes the least squared difference [38, 39] between multispectral image collected for Patient 1 or from a central library (or at Time 1 for surveillance applications) and the multispectral image gathered for the Patient 2 or for the test patient (or at Time 2). An approximate signature S₂ taken at Patient 2 (or Time 2 for surveillance applications) that accounts for global changes in the images by using the “Whitening-DeWhitening” [38, 39] signature transform is estimated as

$${S}_{2} = {m}_{2} + {CVM}_{2}^{\frac{1}{2}} {CVM}_{1}^{- \frac{1}{2}} ( {S}_{1} - {m}_{1} ) \qquad ( 3 )$$

The Whitening-DeWhitening transform (Equation 3) uses statistical information of the image gathered for Patient 2, specifically the background mean m₂ and covariance matrix CVM₂, and similar statistical information of image taken for Patient 1, in particular the Signature S₁, background mean m₁, and covariance CVM₁.

The identification of the pixel depends on the detection threshold set by the user's tolerated maximum false alarm rate or minimum detection rate. A map of candidate targets can then be presented to the image analyst, radiologist or radiation oncologist.

B. Initial Testing Conditions to Test the Feasibility of the Supervised Target Detection Approach

To support the embodiment, the algorithms and approaches were tested on prostate cancer patients. The next few paragraphs [29], [30], [31], [32] describe some the conditions that governed the test. Specifically, the next few sections describe the patient population, the MRI scanning parameters, and the whole mount histology used to assess prostate cancers. The following Sections 1, 2, 3 do not describe the embodiment, merely the testing platform to help verify the efficacy of the approaches described in the embodiment.

1. Study Design and Population

The NIH MRI data of prostate cancer were gathered from The Cancer Imaging Archive (TCIA) [44, 45]. This retrospectively designed, single institution study was approved by the local institutional review board, and was compliant with the Health Insurance Portability and Accountability Act of 1996 [47-47]. Informed consent was obtained from each patient. A total of 45 consecutive patients were enrolled in the study between July 2008 and July 2009. Mean patient age was 60.2 years (median 60, range 49 to 75) and mean PSA was 6.37 ng/ml (median 5.8, range 2.3 to 23.7). All patients had biopsy proven adenocarcinoma of the prostate and mean Gleason score was 6.7 (median 7, range 6 to 9). The inclusion criteria required that robotic assisted radical prostatectomy be performed within 180 days of imaging without any intervening treatment. Exclusion criteria were contraindications to MRI or inability to have an endorectal coil placed.

2. Magnetic Resonance Imaging

The MRI scanning collected DWI, DCE, and structural images in DICOM format. The NIH studies [44-47] used a combination of an endorectal coil (BPX-30, Medrad, Pittsburgh, Pa.) tuned to 127.8 MHz and a 16-channel cardiac coil (SENSE, Philips Medical Systems, Best, The Netherlands) on a 3T magnet (Achieva, Philips Medical Systems) without need for prior bowel preparation. The endorectal coil was inserted using a semi-anesthetic gel (lidocaine) while the patient was in left lateral decubitus position. The balloon surrounding the coil was distended with perfluorocarbon (3 mol/L-Fluorinert, 3M, St. Paul, Minn.) to a volume of approximately 50 ml to reduce susceptibility artifacts induced by air in the coil's balloon. The MRI protocol included triplanar T2W turbo spin echo, DW MRI, 3DMR point resolved spectroscopy, axial pre-contrast T1-weighted axial 3D fast field echo DCE MRI sequences, and their detailed sequence parameters were defined in a prior study[12]. The mean interval between MRI and radical prostatectomy was 60 days (range 3 to 180, median 48). The interval between TRUS-guided biopsy and MRI was 10 or more weeks to avoid post-biopsy hemorrhage related MRI signal changes.

3. Preparation of Customized MRI Based Mold and Histopathological Analysis for NIH Patients

Following MRI, 3D models of each prostate [44-47] for the NIH patients were generated using ANALYZE software (Mayo Clinics, Analyze-Direct, Inc., Overland Park, Kans.). Generation of the 3D model included segmentation of the prostate capsule on in vivo triplane T2W MRI, fusion of the binary objects, and surface extraction of high resolution 3D surfaces from the binary object. Each mold was designed using commercially available 3D computer aided design software (Solidworks, Dassault Systhmes SolidWorks Corp., Concord, Mass.) and the design incorporated the deformation of the endorectal coil. A 3D printer (Dimension Elite 3D printer, Stratasys, Inc., Eden Prairie, Minn.) deposits acrylonitrile butadiene styrene to fabricate each mold. Following robotic radical prostatectomy, the specimen was fixed in formalin for 2 to 24 hours at room temperature, then seminal vesicles were amputated and the specimen was placed in the customized 3D mold and sliced in axial 6 mm sections. This short period of fixation makes the specimen firm and allows slicing without distortion

Whole mount histopathology NIH patient specimens were sectioned in the customized mold and mapped for individual tumor foci, dimensions and Gleason scores independently by 2 experienced pathologists blinded to MRI. Sectioning of the gross specimen in the molds corresponded to the axial plane of the MRI sections. These whole mount sections were processed for histopathology, and paraffin embedded sections were evaluated for the presence and grade of cancer. Foci of cancer were marked on each slide with 2-axis measurements in millimeters. These foci were then mapped on paper.

C. DCE and Time Profile Analysis

This embodiment exploits tumor physiology to help distinguish lesions from normal tissues. Prostate tumors are often highly vascularized. The vasculature is porous to material and the contrast material enters the small extravascular space (but not the cells). Therefore, prostate tumors can fill and empty MRI contrast material quickly relative to normal prostate organ. A simple mathematical two compartment model [48, 49] describes C_{t,}, the tracer concentration in the tissue that supplies and empties through the tumor vasculature. In the model differential equations (Equations 4-5), Cp is the tracer concentration in the plasma, k^{trans} is the transfer rate from plasma to the extra vasculature space, and kₑₚ, is the rate constant describing the return of the tracer from the extra vasculature space to the plasma or washout

$$\frac{d {C}_{t}}{dt} = {k}^{trans} {C}_{p} - {k}_{ep} {C}_{t} \qquad ( 4 )$$

The general solution to the equation is

Cₜ(t)=k^{trans}∫Cₚ(t)exp(−kₑₚ(t−τ)) (5)

To simplify matters, this study analyzed the time profile at times much greater than the peak. Specifically, an exponential tail was fitted using the last 200 seconds of the time profile with an exponentially decaying and constant kep or “washout”.

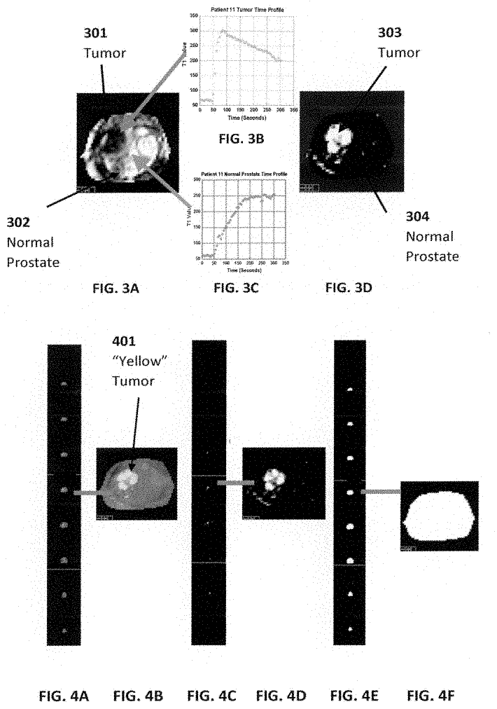

It is conventional to generate Dynamic Contrast Enhancement (DCE) images to help detect prostate cancer. DCE are time series images that follows the evolution of contrast material over several hundred seconds following its injection and uptake in the tissues. FIG. 3A, FIG. 3B, FIG. 3C, FIG. 3D illustrates the analysis of Dynamic Contrast Enhancement images taken from Patient #11. FIG. 3A shows an image of an ADC slice. The dark area in the ADC shows low diffusion and possible tumor 301. FIG. 3B shows time profile from tumor, taken from a single tumor pixel shown within the dark area 301 of FIG. 3A. Note the decay in FIG. 3B for times longer than 100 seconds. FIG. 3C shows time profile from normal prostate 302 taken from a single voxel shown within the bright area in FIG. 3A and shows increasing values, contrasting with FIG. 3B. FIG. 3D is an image of Washout or kₑₚ, shows decay time from analyzing DCE. Bright areas 303 show elevated decay rate and tumor vascularity and dark areas 304 show growth or no growth in time in T1 values.

D. Registration and Mosaicking

Algorithms [36-40] developed for defense applications were modified and extended to handle medical imaging formats using ENVI/IDL, (Harris Geospatial, Melbourne, Fla.). The MRI images were resampled, scaled, translated, resliced and registered at the pixel level to a common spatial resolution (1 mm×1 mm×6 mm) using ENVI/IDL. The processed DCE images were treated as the reference for registration. Resampling in the axial direction was abetted with the patient table positions that were indicated in the MRI. Visual inspection of tissues (such as prostate gland, rectum) and comparing the different modalities provided quality assurance and verification. Occasionally small (1-2 mm) translation of slices were applied in the transverse and axial directions. The multiple axial cubes in three dimensions were “mosaicked” together by sequentially stitching them together into a narrow three dimensional image. In this way, the four dimensions (three dimensional body volume plus the fourth dimension composed of MRI modalities) are compressed into three dimensions using the mosaicked cubes.

FIG. 4 shows an example of the mosaic hypercube and some of its constituents from Patient #11. FIG. 4A is an image of a mosaic is composed of eight slices stitched together. FIG. 4B shows an expanded image of One (out of 7) of the components of the hypercube is a mosaic of the washout or kₑₚ. FIG. 4C is an image of mosaic of kep. FIG. 4D is an image of an expanded view of a single slice from FIG. 4C. The prostate mask for the hypercube FIG. 4E and an expanded view of a single mask slice (FIG. 4F) used to abet the analysis are also shown.

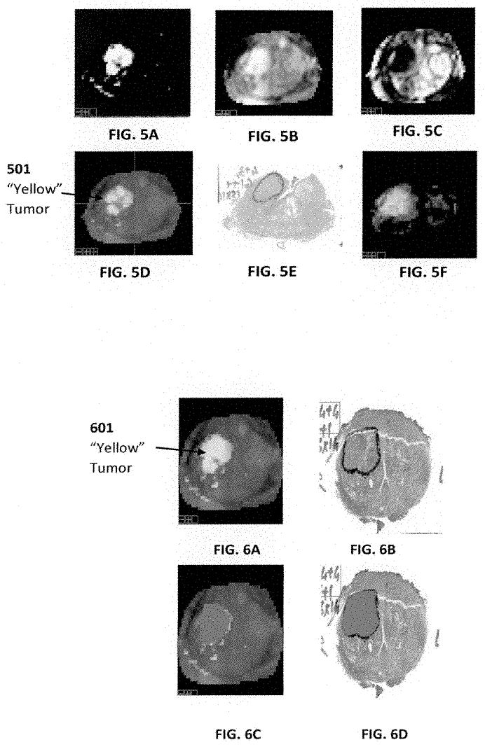

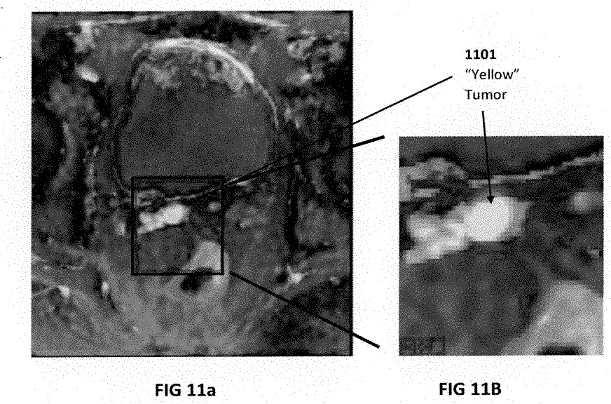

The hypercube is displayed in color by assigning the spatially registered red (R) to washout of kₑₚ, green (G) to DWI-Hi B, blue (B) to ADC. The high tumor vascularity (see Section III.C) will result in elevated kₑₚ or washout (red), reduced proton diffusion from higher cellular density or elevated DWI-High-B (green) but lower ADC (blue). Recall that the high magnetic field gradient (high B value) means the T1 values are elevated for low diffusing protons (i.e. prostate tumor) relative to T1 values from faster diffusing protons. The combination of high red and high green but low blue means the tumor should appear as yellow in color images but denoted as 401 in FIG. 4B, 501 in FIG. 5D, 601 in FIG. 6A, 801 in FIG. 8A, and 1101 in FIGS. 11A and 11B.

E. Multimodality MRI Applications

The technology described in this embodiment has been applied to a series of TCIA Prostate Cancer patients imaged at NIH. For example, FIGS. 5A, 5B, and 5C display the slices of Washout or kₑₚ, DWI High-B (B=1000 sec/mm²), and ADC for Patient #11, respectively for prostate in the TCIA NIH series. An ACE map of prostate tumor for Patient #11 in the NIH Series is shown (FIG. 5F) as pseudo color image of a single (1 out of 10) slice. The ACE detection used seven modalities (Washout, Fit Probability to exponential decay, T1 (pre-contrast injection), T1 (highest contrast uptake), T2, DWI-Highest B or largest magnetic field gradient and ADC. Washout and Fit Probability modalities are derived from analysis of the DCE images. The tumor appears in the right mid peripheral zone of the prostate (FIGS. 5A,5B,5C,5D,5F), in agreement with the histological analysis (FIG. 5E).

F. Tumor Volume Measurements

Measuring the tumor volume is a critical component for assessing the patient's condition and for help to guide treatment decisions. Color displays (tumor shown as yellow as discussed above, shown in FIG. 6A and denoted as 601) and ACE detection (also discussed above) delineate the tumor. The color displays were generated by assigning red, green, and blue channels to the grey scale images of the washout or kₑₚ, DWI, and ADC images respectively. The number of pixels inside the yellow portion of the RGB images (or Region of Interest) can be determined from standard image processing (see red area inside the contoured image, FIG. 6C). In addition, to test these ideas, a matching tumor outlined by a pathologist (see for before FIG. 6B, and after FIG. 6D) outlined histology images). A comparison of tumor areas in each slice can be made between the MRI and the histology images after accounting for different spatial resolutions for the two sets of images. In FIG. 6A, 6C, the spatial resolution is Imm per pixel for the registered MRI image set and 47 pixels per mm for the histology images (FIG. 6B, 6D) (scaling of 47×47=2209). To convert to tumor volume, both sets of images have a slice thickness of 6 mm. and therefore the tumor volume conversion factor going from histology to MRI is 1/2209. The tumor areas for the single displayed slice were measured to contain 239 and 323,196 pixels in the MRI (FIG. 6A, 6C) and histology images (FIG. 6B, 6D), respectively. Converting the histology results in 145 pixels, rather than 239.

To validate the tumor volume measurement approach advanced in this embodiment, there is an additional correction required for comparing with the histologically derived tumor measurements. Formalin fixation is required to generate the histological slices and has been observed to shrink the prostate and tumors. The histology derived volumes must be corrected for shrinkage for the histology slices by a factor 1.15 [56-58].

G. Gleason Scoring

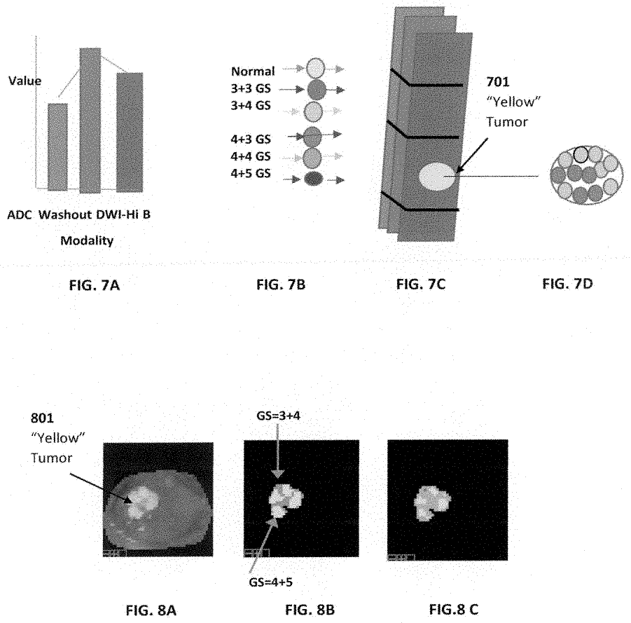

FIG. 7A, FIG. 7B, FIG. 7C, FIG. 7D illustrates the process for generating hypercube mosaic from the MRI images. To non-invasively Gleason score tumors, a battery of signatures (FIG. 7A) of normal tissue (3 normal prostate tissues) and various tumor signatures (3+3, 3+4, 4+3, 4+4, 4+5, 5+5) (FIG. 7B) were inserted into the ACE and SAM (Spectral Angle Mapper) supervised target searches. In addition, the signatures were transformed using the Whitened-DeWhitened transform (Equation 3). This transform is intended to account for global changes (such as differences in pulse sequences, image calibration etc.) and relies on statistics such as the mean (m), covariance (R) of the multispectral image. The signatures (FIG. 7A, 7B) are taken from the NIH/TCIA prostate tumor cohort. The Gleason score in histology images was identified from each patient and slice. A hypercube (FIG. 7C) of 9 dimensions (3 normal tissue plus 6 Gleason scores) composed of all these ACE/SAM detection scores was generated with the aid of custom designed ENVI/IDL software. Due to the large tumor size in some of the patients, the tumor itself could affect the statistics that describe the prostate. To reduce that error in determining the background or normal prostate statistics, the tumor was digitally masked or removed from the prostate when calculating the mean and covariance used in the transform.

Each voxel inside the tumor (FIG. 7D) was examined to find which signature delivered the highest ACE/SAM value. In addition, the top two generating target detections were recorded and weighted based on their ACE/SAM values. The tumor area was identified and the mean and standard deviation for the maximum and weighted ACE/SAM scores were recorded. A total of four types of scoring were applied to each pixel within the tumor: Maximum SAM, Maximum SAM using the Whitening-DeWhitening transform, Weighted Top two Gleason scorers (untransformed signatures), and Weighted Top two Gleason scorers using the Whitening-DeWhitening transform.

FIG. 8 shows an example of Gleason scoring output. A color display (red assigned to kep, green to DWI-High B, blue to ADC) of a prostate from Patient #11 is shown in FIG. 8A. FIG. 8B shows the Gleason scoring within the tumor using Whitened-DeWhitened signatures applied to SAM and the maximum SAM is recorded for each pixel. Similarly, FIG. 5C shows the Gleason scoring within the tumor using Whitened-DeWhitened signatures applied to SAM and the top two weighted SAM scores are recorded for each pixel. The false coloring scheme depicting the Gleason Scores in FIG. 8C is less distinct than FIG. 8B due to the weighting of the detected Gleason scores.

H. Metastases Detection and Display

The techniques described in this embodiment, specifically supervised target detection, Whitening-Dewhitening of target signatures, and color display to highlight tumors, can also be applied to tissues that reside outside the prostate. Specifically, these techniques can be applied to find metastases such as detecting possible seminal vesicle involvement. The only caveat is that to accurately apply the Whitening-Dewhitening transform for target signatures requires using background statistics for the mean (m) and covariance matrix (CVM) of common tissues imaged in both sets of MRI scans (such as normal prostate tissue) for the Time 1 (Library) and the Time 2 (Test) patients. To apply the supervised target detection such as ACE requires the background that is used to find the mean (m) and covariance matrix (CVM) must include tissues that are under investigation that may not include the prostate tumor.

### IIII. Results/Support for the Embodiment

Ten sets of prostate tumor volumes from the MRI and Histology images were analyzed. These ten patients were selected for proof of principal because they showed evident washout rates (kₑₚ) within the prostate indicating high vascularization within the tumor. No restrictions were placed regarding tumor placement in the peripheral zone or central gland, nor size (1 cc to 15 cc). FIG. 9 shows a plot of the histology volume against the MRI volumes. A linear fit was applied to the data showing high correlation coefficient (R=0.94, P=0.0005), high fitted slope (0.78) and low intercept (0.5). Tumors can be quite heterogeneous so that a single signature inserted into a supervised target detector such as ACE and SAM may not detect the entire tumor. Multiple signatures may need to be inserted into ACE and SAM to cover the entire tumor. Moreover, it is likely that the “radiologic margin” is less sensitive than the “histologic margin” due to decreases in tumor density in the advancing edge of the tumor.

The histology derived volumes were corrected for shrinkage for the histology slices using a factor of 1.15 [56-58]. Correcting all tumor volumes taken from radical prostatectomy in this embodiment results in a correlation coefficient of R=0.94 when plotted against the measurements derived from supervised target detection and multiparametric MRI. Using the shrinkage factor, the slope for the tumor volumes histology vs multiparametric MRI increased to 0.904+/−0.115 (Standard Error), consistent with a slope of 1.0. The fitted intercept is found to be 0.631+/−0.881 (Standard Error), consistent with an intercept 0.0.

The Gleason scores (GS) for the same set of patients were analyzed and compared to the scores determined by the pathologist's assessment of histology slices. This study examined tumors throughout the prostate and attempted to minimize the bias by not confining the search to the peripheral zone nor to the central gland. The GS algorithm queried the SAM value (Equation 2), found the maximum GS and also a weighted average of the top two GS's. Four sets of measurements were recorded for each voxel inside the tumor: Maximum SAM, Weighted Average SAM using untransformed signatures, and Maximum SAM, and Weighted Average SAM using transformed signatures.

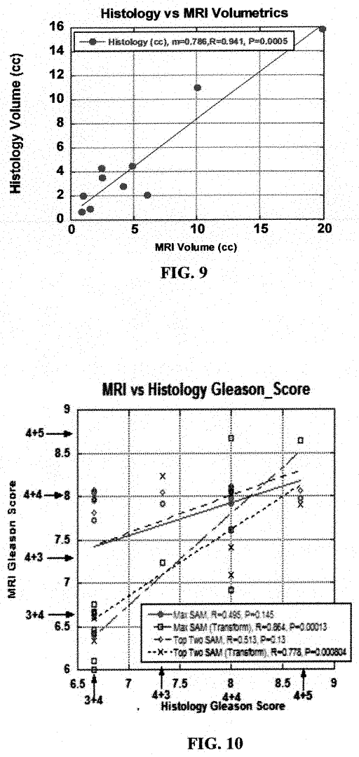

FIG. 10 plots the average GS within a tumor using a number of ACE and SAM target detection algorithms applied to registered MRI against the pathologist's assessment. The highest correlation (R=0.86, P=0.00012) and highest slope is generated using the Maximum SAM values and transformed signatures as shown in FIG. 10. Conventional Gleason scores are superimposed on both sets of axis in FIG. 10. As is evident, there is some inter-patient global variability in the image generation that is corrected using the signatures with the Whitening-DeWhitening transform.

Other methods have generated similar overall results, but this method produces results on a per voxel basis, making it more useful for directing biopsies and opens up the possibility for local treatments (external beam radiation therapy, brachytherapy, cryotherapy) to target areas of disease and not just treat the whole organ, which is often associated with morbidity and decreases in patient quality of life. Supervised target detection achieved high correlation scores, ranging from 0.78 to 0.94, between the predicted and actual Gleason scores.

Prostate Tumor staging requires determining whether the cancer as metastasized to seminal vesicles. To demonstrate the feasibility for finding tumors residing outside the prostate, metastases was observed in a few cases using color approach that was previously discussed. FIG. 11A shows the application of red, green, blue colors to the registered Washout, DWI (High B), and T2 components of the hypercubes, respectively. An expanded view of FIG. 11A is shown in FIG. 11B. The tumor in the seminal vesicle appears yellow in the color image but denoted as 1101 in the black and white image, due to the combination of reduced diffusion, elevated emptying rates, and relatively low T2 values.

Although the present invention has been described in connection with embodiments thereof, it will be appreciated by those skilled in the art that additions, deletions, modifications, and substitutions not specifically described may be made without departure from the spirit and scope of the invention as defined in the appended claims.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically “adapted/adaptable,” “able to,” “conformable/conformed to,” etc. Those skilled in the art will recognize that such terms (e.g., “configured to”) can generally encompass active state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as“including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically interacting, and/or logically interactable components.

In some instances, one or more components may be referred to herein as “configured to,” “configured by,” “configurable to, “operable/operative to,” means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The illustrative embodiments described in the detailed description, drawings, and claims are not meant to be limiting. Other embodiments may be utilized, and other changes may be made, without departing from the spirit or scope of the subject matter presented here.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken as limiting.

### V. References

[1] R. L. Siegel, K. D. Miller, A. Jemal. “Cancer statistics, 2015”. CA Cancer J Clin. Vol. 66(1), pp. 7-30, 2016.
[2] S. E. Eggener, et al. “Predicting 15-year prostate cancer specific mortality after radical prostatectomy”. J Urol. Vol. 185(3), pp. 869-875, 2011.
[3] H. J. Lavery et al. “Gleason patterns 3 and 4 prostate cancer represent separate disease states?” J Urol. Vol. 188(5), pp. 1667-1675, 2012.
[4] C. R. King. J. P. Long “Prostate biopsy grading errors: A sampling problem?” Int J Cancer. Vol. 90(6), pp. 326-330, 2000.
[5] J. I. Epstein et al. “Upgrading and downgrading of prostate cancer from biopsy to radical prostatectomy: Incidence and predictive factors using the modified Gleason grading system and factoring intertiary grades”. Eur Urol. Vol. 61(5), pp. 1019-1024, 2012.
[6] R. K. Berglund, et al. “Pathological upgrading and up staging with immediate repeat biopsy in patients eligible for active surveillance”, J Urol., Vol. 180(5), pp. 1964-1967, discussion 1967-1968, 2008.
[7] J. Wu and M. L. Gonzalgo, “Use of Magnetic Resonance Imaging to Accurately Detect and Stage Prostate Cancer. The Hype and the Hope”. J Urol. Vol. 186, pp. 1756-1757, 2011.
[8] A. R. Padhani et al., “Dynamic Contrast-Enhanced MRI in Clinical Oncology: Current Status and Future Directions”, J. Magn Res Imag. Vol. 16, pp. 407-422, 2002.
[9] C. K. Kim et al. “Value of diffusion-weighted imaging for the prediction of prostate cancer location at 3T using a phased-array coil: preliminary results”, Invest Radiol, Vol. 42 (12), pp. 842-847, 2007.
[10] H. A. Vargas, et al., “Diffusion-weighted endorectal MR imaging at 3 T for prostate cancer: Tumor detection and assessment of aggressiveness”, Radiology. Vol. 259(3) pp. 775-784, 2011.
[11] C. Sato, et al., “Differentiation of noncancerous tissue and cancer lesions by apparent diffusion coefficient values in transition and peripheral zones of the prostate”, J Magn Reson Imaging, Vol. 21(3) pp. 258-262, 2005.
[12] J. V. Hegde et al., “Multiparametric MRI of Prostate Cancer: An Update on State-of-the-Art Techniques and Their Performance in Detecting and Localizing Prostate Cancer”, J. Mag Res Imaging, Vol. 37:1035-1054, 2013.
[13] J. J Fütterer, et al. “Prostate cancer localization with dynamic contrast-enhanced MR imaging and proton MR spectroscopic imaging”, Radiology, Vol. 241(2) pp. 449-458, 2006.
[14] A. Tanimoto et al. “Prostate cancer screening: The clinical value of diffusion-weighted imaging and dynamic MR imaging in combination with T2-weighted imaging”. J Magn Reson Imaging, Vol. 5(1), pp. 146-152, 2007.
[15] K. Kitajima, et al. “Prostate cancer detection with 3 T MRI: Comparison of diffusion-weighted imaging and dynamic contrast-enhanced MRI in combination with T2-weighted imaging”, J Magn Reson Imagi. 2010; Vol. 31(3), pp. 625-31, 2010. doi: 10.1002/jmri.22075.
[16] D. L. Langer et al., “Prostate Cancer Detection With Multi-parametric MRI: Logistic Regression Analysis of Quantitative T2, Diffusion-Weighted Imaging, and Dynamic Contrast-Enhanced MRI”. J. Mag Res Imag, Vol. 30, pp. 327-334, 2009.
[17] P. C. Vos et al. “Automatic computer-aided detection of prostate cancer based on multiparametric magnetic resonance image analysis”, Med. Biol., Vol. 57, pp. 1527-1542, 2012.
[18] M. A. Haider et al. “Combined T2-Weighted and Diffusion-Weighted MRI for Localization of Prostate Cancer”. AJR., Vol. 189, pp. 323-328, 2007.
[19] P. C. Vos et al. “Computer-assisted analysis of peripheral zone prostate lesions using T2-weighted and dynamic contrast enhanced T1-weighted MRI”. Phys. Med. Biol., Vol. 55, pp. 1719-1734, 2010.
[20] B. Turkbey et al., “Multiparametric 3T Prostate Magnetic Resonance Imaging to Detect Cancer: Histopathological Correlation Using Prostatectomy Specimens Processed in Customized Magnetic Resonance Imaging Based Molds”, J. Urol., Vol. 186: pp. 1818-1824, 2011.
[21] J. S Isebaert et al. “Multiparametric MRI for Prostate Cancer Localization in Correlation to Whole-Mount Histopathology”, Magn. Reson. Imaging., Vol. 37, pp. 1392-1401, 2013.
[22] B. Turkbey et al.” Prostate Cancer: Value of Multiparametric MR Imaging at 3 T for Detection-Histopathologic Correlation”, Radiology, Vol. 255, pp. 89-99, 2010.
[23] G J. Metzger et al. “Detection of Prostate Cancer: Multiparametric MR Imaging Models Developed by Using Registered Correlative Histopathologic Results”, Vol. 279: pp. 805-816, 2016 10.1148/radiol.2015151089.
[24] O. F. Donati et al. “Prostate cancer aggressiveness: Assessment with whole-lesion histogram analysis of the apparent diffusion coefficient”. Radiology. Vol. 271(1), pp. 143-152, 2014.
[25] O. F. Donati, et al. “Prostate MRI: Evaluating tumor volume and apparent diffusion coefficient as surrogate biomarkers for predicting tumor Gleason score”. Clin Cancer Res. Vol. 20(14), pp. 3705-3711, 2014.
[26] N. M deSouza, et al.,” Diffusion-weighted magnetic resonance imaging: A potential non-invasive marker of tumour aggressiveness in localized prostate cancer” Clin Radiol. Vol. 63(7), pp. 774-782, 2008.
[27] K. Shigemura et al., “Can Diffusion-Weighted Magnetic Resonance Imaging Predict a High Gleason Score of Prostate Cancer?” Korean J Urol. Vol. 54, pp. 234-238, 2013.
[28] Y. Mazaheri, et al., “Prostate cancer. Identification with combined diffusion weighted MR imaging and 3D 1H MR spectroscopic imaging-correlation with pathologic findings”, Radiology. Vol. 246(2), pp. 480-488, 2008.
[29] D. L. Langer, et al. “Prostate tissue composition and MR measurements: Investigating the relationships between ADC, T2, K(trans), v(e), and corresponding histologic features”, Radiology. Vol. 255(2), pp. 485-494, 2010.
[30] A. Oto, et al. “Diffusion-weighted and dynamic contrast-enhanced MRI of prostate cancer: Correlation of quantitative MR parameters with Gleason score and tumor angiogenesis”, AJR Am J Roentgenol Vol. 197(6), pp. 1382-1390, 2011.
[31] Y. Peng et al. “Quantitative analysis of multiparametric prostate MR images: Differentiation between prostate cancer and normal tissue and correlation with Gleason score—a computer-aided diagnosis development study”, Radiology, Vol. 267(3), pp. 787-796, 2013.
[32] M. Moradi et al. “Multiparametric MRI maps for detection and grading of dominant prostate tumors”, J Magn Reson Imaging”, Vol. 35(6), pp. 1403-1413, 2012.
[33] M. M. Siddiqui et al., “Prediction of prostate cancer Gleason score using a MRI-based nomogram”, J Clin Oncol. Vol. 32: (suppl 4; abstr 255), 2014.
[34] F. Citak-Er et al. “Final Gleason Score Prediction Using Discriminant Analysis and Support Vector Machine Based on Preoperative Multiparametric MR Imaging of Prostate Cancer at 3T”, BioMed Research International Volume 2014, Article ID 690787, 9 pages http://dx.doi.org/10.1155/2014/690787
[35] D. Fehra et al. “Automatic classification of prostate cancer Gleason scores from multiparametric magnetic resonance images”. PNAS. Vol. 112: pp. E6265-E6273 2015 doi: 10.1073/pnas.1505935112.
[36] J. A Richards, X. Jia, Remote Sensing Digital Image Analysis, New York: Springer-Verlag, 1999.
[37] D. Manolakis, G. Shaw, “Detection algorithms for hyperspectral imaging applications”, IEEE Signal Processing Magazine. 2002; Vol. 19, pp. 29-43, 2002.
[38] R. Mayer et al, “Object Detection and Color Constancy Using a Whitening Transformation in Multi-spectral Imagery”. 2002 MSS Specialty Group on Joint Passive Sensors/CCD, Vol. 1 2002.
[39] R. Mayer et al. “Object Detection by Using “Whitening/DeWhitening” to Transform Target Signatures in Multi-temporal Hyper- and Multi-spectral Imagery”, IEEE Transactions Geoscience and Remote Sensing. Vol. 41, pp. 1136-1142, 200.
[40] J. M Schuler et al. “Robust color fusion of diurnal multi-spectral imagery: Empirical Color Constancy”. 2002 MSS Specialty Group on Joint Passive Sensors/CCD, Vol. 1, 2002.
[41] Weinreb J C et al., “PI-RADS Prostate Imaging-Reporting and Data System: 2015, Version 2”. Eur Urol Vol. 69(1), pp. 16-40, 2016
[42] S. E. Viswanath et al. “Central gland and peripheral zone prostate tumors have significantly different quantitative imaging signatures on 3 Tesla endorectal, in vivo T2-weighted MR imagery”, J Magn Reson Imaging. Vol. 36(1) pp. 213-224, 2012.
[43] P. Tiwari et al. “Multimodal wavelet embedding representation for data combination (MaWERiC): integrating magnetic resonance imaging and spectroscopy for prostate cancer detection”, NMR Biomed Vol. 25(4), pp. 607-619, 2012.
[44]K. Smith et al. Data From Prostate-MRI. http://dx.doi.org/10.7937/K9/TCIA.2016.6046GUDv42
[45] K. Clark et al. The Cancer Imaging Archive (TCIA): Maintaining and Operating a Public Information Repository, Journal of Digital Imaging, Vol. 26 (6), pp. 1045-1057, 2013.
[46] V. Shah et al., “A method for correlating in vivo prostate magnetic resonance imaging and histopathology using individualized magnetic resonance-based molds” Rev Sci Instrum. 0(10):104301, 2009.
[47] B. Turkbey et al. “Multiparametric 3T prostate magnetic resonance imaging to detect cancer: histopathological correlation using prostatectomy specimens processed in customized magnetic resonance imaging based molds.” J Urol. Vol. 186(5), pp. 1818-24, 2011.
[48] P. S. Tofts et al., “Estimating Kinetic Parameters from Dynamic Contrast-EnhancedTl-Weighted MRI of a Diffusable Tracer: Standardized Quantities and Symbols”, J Magn Res Imag. Vol. 10, pp. 223-232, 1999.
[49] P. S. Toft, “T1-weighted DCE Imaging Concepts: Modelling, Acquisition and Analysis”, Magnetom Flash; Vol. 3: pp. 31-39, 2010.
[50] E. S. Wisenbaugh et al., “Proton Beam Therapy for Localized Prostate Cancer 101: Basics, Controversies, and Facts” Rev Urol. Vol. 16(2), pp. 67-75, 2014.
[51] T. Zilli et al. “Urethra-sparing, intraoperative, real-time planned, permanent-seed prostate brachytherapy, toxicity analysis”, Int. J Rad Onc_Biol Phys. Vol. 81(4), pp. e377-e383, 2011.
[52] J. Vainshtein et al. “Randomized phase II trial of urethral sparing intensity modulated radiation therapy in low-risk prostate cancer: implications for focal therapy”. Rad Onc. Vol. 7, pp. 82-90, 2012.
[53] T. Zilli et al. “SBRT—Dosimetric results, randomized phase II trial, urethra-sparing SBRT for localized prostate cancer” Int. J Rad Onc_Biol Phys. Vol. S900 ASTRO 3792 2014.
[54] M. Fager et al. “Linear energy transfer painting with proton therapy: a means of reducing radiation doses with equivalent clinical effectiveness”, Int J Radiat Oncol Biol Phys. Vol. 91(5): pp. 1057-64, 2015.
[55] E. Niaf et al. “Kernel-based learning from both qualitative and quantitative labels: application to prostate cancer diagnosis based on multiparametric MR imaging”, IEEE Trans on Image Process Vol. 23 (3) pp. 979-91, 2014.
[56] B. Turkbey, H. Mani, O Aras, A. R. Rastinehad, V. Shah, M. Bernardo, T. Pohida, D. Daar, C.

Benjamin, Y. L. McKinney, W. M. Linehan, B. J. Wood, M. J. Merino, P. L. Choyke P. A. Pinto, “Correlation of Magnetic Resonance Imaging Tumor Volume with Histopathology,” J. Urol Vol. 188, pp. 1157-1163, 2012.
[57] G J. Jager, E. T. Ruijter, C. A. van de Kaal, “Local staging of prostate cancer with endorectal MR imaging: correlation with histopathology”, AJR Vol. 166 pp. 845, 1996.
[58] S. Jonmarker, A. Valdman, A. Lindberg, “Tissue shrinkage after fixation with formalin injection of prostatectomy specimens”, Virchows Arch, Vol. 449: 297, 2006.

1: A method to non-invasively determine the tumor aggressiveness without using needle biopsies.
A. Generating digitally magnetic resonance images of patients with possible tumors wherein the MRI scanning conditions are similar for all patients and wherein all digital image sets include DWI (Diffusion Weighted Images), DCE (Dynamic Contrast Enhancement), and structural (T1, T2) images.
B. Processing digitally by using Custom Software (CS) said images (claim 1.A) to digitally extract Washout from said DCE (claim 1.A) and Apparent Diffusion Coefficient from said DWI (claim 1.A) images and possibly other images.
C. Resampling, altering transverse spatial resolution, and reslicing digitally by using Commercial Off the Shelf (COTS) of the said patient MRI images (claim 1.B) to a common spatial resolution (for example 1 mm, 1 mm, 6 mm for x, y, z directions).
D. Repositioning slices in axial direction using table positions from said MRI by applying the COTS for hyperspectral image and wavelength resampling and interpolation taken from ENVI Software.
E. Translating and registering digitally by using either COTS or CS for each of the said MR images (claim 1.C) to the voxel level with the aid of common anatomical structures in said MR images to guide and digitally create a hypercube.
F. Using CS to sequentially digitally stitching together said multiple axial hypercubes cubes (claim 1.D) from each slice to form a mosaic of hypercubes for each patient.
G. Creating, by using CS digital In-Scene signatures or image-based biomarkers derived from a training set (that have concomitant histopathologic assessments from whole mount radical prostatectomy) from said mosaicked, registered patient image hypercubes (claim 1.E).
H. Inserting digitally by using CS In-Scene signatures into Adaptive Cosine Mapper applied to said mosaicked registered patient hypercubes (claim I.E) to create target detection maps depicting tumors in every slice.
I. Contouring digitally by using COTS prostate organ of said mosaicked hypercubes MR (claim 1.E) to create prostate organ mask.
J. Generating digitally by using COTS pure prostate mask by subtracting said tumor mask (claim 1.G) from said prostate mask (claim 1.H).
K. Creating digitally by using COTS Gleason Score signatures or image-based biomarkers using a training set from said mosaicked patient image cubes (claim 1.E) and correlated with histopathology analysis of Gleason Scores of the patient.
L. Creating digitally by using CS a battery of signatures or image-based biomarkers depicting Gleason Scores from said signatures (claim 1.J) ranging from normal tissue to 3+3, 3+4, 4+3, 4+4, 4+5, 5+4, 5+5 by comparing with concomitant histopathological assessments from whole mount radical prostatectomy.
M. Inserting digitally by using CS said battery of untransformed signatures (claim 1.K) into supervised target detection algorithms such as Adaptive Cosine Estimator (ACE) and Spectral Angle Mapper (SAM) with conical decision surfaces that use background of pure normal prostate for background mean and covariance statistics and applying computation to every voxel in said tumor (claim 1.G).
N. Examining digitally by using CS each voxel inside the said tumor to determine which pixel achieves the highest detection (claim 1.L) of a given Gleason score.
O. Computing and recording by using CS digital Gleason score mean and standard deviation of from said searches inside tumor (claim 1.L).
Whereby the said prostate tumor's (claim 1.L) average and standard deviation for the Gleason Score is found using untransformed signatures or image-based biomarkers biomarkers inserted into supervised target detection algorithms to determine Gleason score non-invasively.
P. Inserting digitally by using CS said battery of signatures or image-based markers (claim 1.K) and applying said pure prostate mask (claim 1.I) to mosaicked hypercube (claim 1.E) for help in calculating background statistics for transforming said signatures based on Whitening Dewhitening transform.
Q. Inserting digitally by using CS said Whitened-Dewhitened transformed signatures (claim 1.O) into ACE and/or SAM transform and applying computation to every voxel in said tumor (claim 1.G).
R. Examining digitally by using CS to query each voxel inside the said tumor (claim 1.G) to determine which Gleason score achieves the highest detection level (claim 1.P).
S. Computing and recording digitally by using CS mean and standard deviation of Gleason Scores (claim 1.Q) from said searches inside tumor (claim 1.G).
Whereby the said prostate tumor's (claim 1.L) average and standard deviation for the Gleason Score using transformed (Whitened-DeWhitened) signatures or image-based biomarkers biomarkers inserted into supervised target detection algorithms determines Gleason score non-invasively.

2: A simple method to display tumors and normal tissues using color (not pseudo-color).
A. Assigning digitally by using COTS red (r) to washout of kep, green (g) to DWI-Hi B, blue (b) to ADC to said registered hypercube (claim 1.E).
1. The hypercube is displayed in color by assigning the spatially registered images. The combination of high red and high green but low blue means the tumor should appear as yellow.
Whereby the said prostate tumor and prostate are highlighted and displayed in color.

3: A method to non-invasively determine the tumor volume.
Method #1
A. Identifying the color of yellow in said colored mosaicked hypercube (claim 2.A).
B. Counting digitally by using COTS yellow pixels in said mosaicked hypercube. (claim 3.A).
A. Computing digitally by using COTS tumor volume by using number of said pixels (claim 3.B). exceeding a certain level of yellow and inserting into tumor volume=# of pixels exceeding yellow threshold×1 mm×1 mm×6 mm (in this sample, for illustrative purposes) or the common resolution in all three dimensions (common transverse x direction resolution X common transverse y resolution X common z axial direction resolution).
Whereby the said prostate tumor's (claim 1.L) volume is determined non-invasively.
Method #2
B. Choosing threshold for said tumor (claim 1.G) in hypercube.
C. Counting digitally by using COTS number of pixels in said mosaicked hypercube (claim 1.G) exceeding said threshold (claim 3D).
D. Computing digitally by using COTS tumor volume by using number of said pixels exceeding threshold (claim 3.E) and inserting into tumor volume=# of pixels exceeding threshold×1 mm×1 mm×6 mm or the common resolution in all three dimensions (common transverse x direction resolution×common transverse y resolution×common z axial direction resolution).
Whereby the said prostate tumor's (claim 1.L) volume is determined non-invasively.

4: A simple method to display metastases and normal tissues using color (not pseudo-color).
A. Assigning digitally by using COTS red (r) to washout of kₑₚ, green (g) to DWI-Hi B, blue (b) to ADC to said registered hypercube (claim 1.E).
1. The hypercube is displayed in color by assigning the spatially registered images. The combination of high red and high green but low blue means the metastases should appear as yellow.
Whereby the said metastases and normal tissues are highlighted and displayed in color.

5: A method to non-invasively find metastases without using needle biopsies.
Method #1
A. Generating digitally magnetic resonance images of patients with possible metastases wherein the MRI scanning conditions are similar for all patients and wherein all digital image sets include DWI (Diffusion Weighted Images), DCE (Dynamic Contrast Enhancement), and structural (T1, T2) images.
B. Processing digitally by using Custom Software (CS) said images (claim 5.A) to digitally extract Washout from said DCE (claim 5.A) and Apparent Diffusion Coefficient from said DWI (claim 5.A) images and possibly other images.
C. Resampling, altering transverse spatial resolution, and reslicing digitally by using Commercial Off the Shelf (COTS) of the said patient MRI images (claim 5.B) to a common spatial resolution (for example 1 mm, 1 mm, 6 mm for x, y, z directions).
D. Repositioning slices in axial direction using table positions from said MRI by applying the COTS for hyperspectral image and wavelength resampling and interpolation taken from ENVI Software using Table position of MRI and COTS to interpolate images.
E. Translating and registering digitally by using either COTS or CS for each of the said MR images (claim 5.C) to the voxel level with the aid of common anatomical structures in said MR images to refine and guide and digitally create a hypercube.
F. Using CS to sequentially digitally stitching together said multiple axial hypercubes cubes (claim 5.D) from each slice to form a mosaic of hypercubes for each patient.
G. Creating, by using CS digital In-Scene signatures or image-based biomarkers derived from a training set that have concomitant histopathologic assessments from whole mount radical prostatectomy to help identify signatures from said mosaicked, registered patient image hypercubes (claim 5.F).
H. Inserting digitally by using CS In-Scene signatures (claim 5.G) into Adaptive Cosine Estimator Mapper applied to said mosaicked registered patient hypercubes (claim 5.F) using statistics of tissues into create target detection maps in every slice and possibly depicting possible tumor metastases at the voxel level.
Whereby the said metastases (claim 5.H) is non-invasively found at the voxel level using untransformed signatures or image-based biomarkers inserted into supervised target detection algorithms (Adaptive Cosine Estimator).
Method #2
I. Contouring digitally by using COTS prostate organ of said mosaicked hypercubes MR (claim 5.F) to create prostate organ mask.
J. Generating digitally by using COTS pure prostate mask by subtracting said tumor mask (claim 5.H) from said prostate mask (claim 5.I) to generate normal prostate mask.
K. Applying normal prostate mask (claim 5.J) to hypercube (claim 5.F) to calculate background statistics for Time 1 (Library) and Time 2 (test) patients for Whitening-DeWhitening transform.
L. Inserting digitally by using CS said signatures or image-based markers (claim 5.G) and applying said pure prostate mask (claim 5.J) to mosaicked hypercube (claim 5.F) for calculating background statistics for transforming said signatures based on Whitening-Dewhitening transform.
M. Inserting digitally by using CS said Whitened-Dewhitened transformed signatures (claim 5.L) into ACE and/or SAM transform and applying computation to every voxel in said mosaicked hypercube (claim 5.F) to generate metastases detection.
Whereby the said detection of metastases (claim 5.M) at the voxel level using transformed (Whitened-DeWhitened) signatures or image-based biomarkers and supervised target detection (ACE) applied to Time 2 or test mosaicked hypercube (claim 5.F).
